(19) United States
(12) Patent Application Publication
(10) Pub. No.: US 2024/0018258 A1
(43) Pub. Date: Jan. 18, 2024

(54) CD47 BLOCKADE THERAPY WITH CD38 ANTIBODY

(71) Applicant: Pfizer Inc., New York, NY (US)

(72) Inventors: Gloria Hoi Ying LIN, Oakville (CA); Natasja Nielsen VILLER, Etobicoke (CA); Mark Michael WONG, Toronto (CA); Jeffrey Todd WINSTON, Mississauga (CA); Robert Adam UGER, Richmond Hill (CA)

(73) Assignee: Pfizer Inc., New York, NY (US)

(21) Appl. No.: 18/458,692

(22) Filed: Aug. 30, 2023

Related U.S. Application Data
(63) Continuation of application No. 16/979,707, filed on Sep. 10, 2020.
(60) Provisional application No. 62/642,131, filed on Mar. 13, 2018.

(51) Int. Cl. C07K 16/28; A61P 35/00; C07K 14/735

(52) U.S. Cl. CPC C07K 16/2896; A61P 35/00; C07K 14/70535

(57) ABSTRACT

CD47+ disease cells such as cancer cells are treated using a combination of CD47 blocking agent and a CD38 antibody such as daratumumab. The anti-cancer effect of SIRPαFc is enhanced in the presence of daratumumab. Specific combinations include SIRPαFc forms that comprise an Fc that is either IgG1 or IgG4 isotype. These combinations are useful particularly to treat blood cancers including lymphomas, leukemias and myelomas.

### FIELD

This disclosure relates to methods and uses of a drug that blocks the CD47/SIRPα interaction. More particularly, the disclosure relates to methods and uses that, in combination, are useful for improving cancer therapy.

### BACKGROUND

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα to treat cancer by CD47 blockade is described in WO2010/130053.

Trillium Therapeutics' WO2014/094122 describes a protein drug that inhibits or antagonizes interaction between CD47 and SIRPα. This CD47 blocking agent is a form of human SIRPα that incorporates a particular region of its extracellular domain, linked with a particularly useful form of an IgG-based Fc region. In this form, the SIRPαFc drug shows dramatic effects on the viability of cancer cells that present with a CD47+ phenotype. The effect is seen particularly on acute myelogenous leukemia (AML) cells, and many other types of cancer. A soluble form of SIRP having significantly altered primary structure and potent CD47 binding affinity is described in WO2013/109752.

Other CD47 blocking agents have been described, and these include various CD47 antibodies (see for instance Stanford's U.S. Pat. No. 8,562,997, and InhibRx' WO2014/123580), each comprising different antigen binding sites but having, in common, the ability to compete with endogenous SIRPα for binding to CD47, to interact with macrophages and, ultimately, to increase CD47+ disease cell depletion. These CD47 antibodies have activities in vivo that are quite different from those intrinsic to drugs that incorporate SIRPα structure. The latter, for instance, display negligible binding to red blood cells whereas the opposite property in CD47 antibodies, and in high affinity SIRPα variants, creates a need for strategies that accommodate a drug “sink” that follows administration.

Still other agents are proposed for use in blocking the CD47/SIRPα axis. These include CD47Fc proteins described in Viral Logic's WO2010/083253, and SIRPα antibodies as described in University Health Network's WO2013/056352, Eberhard's U.S. Pat. No. 6,913,894, and elsewhere.

The CD47 blockade approach in anti-cancer drug development shows great clinical promise. There is a need to provide methods and means for improving the effect of these drugs, and in particular for improving the effect of the CD47 blocking agents that incorporate CD47-binding forms of SIRPα.

### SUMMARY

The effect of an anti-tumour antibody is enhanced when combined with a CD47 blocking agent. This disclosure reveals that the anti-cancer effect of SIRPαFc, in particular, is enhanced when administered in combination with a CD38 antibody. In embodiments, the SIRPαFc has an IgG4 isotype that comprises an IgV domain of human SIRPα, and the CD38 antibody is daratumumab. The enhancement of daratumumab activity caused by SIRPαFc manifests as an increased depletion of treated CD47+ cancer cells, as an improvement in patient survival, and/or as a reduction in tumour size or distribution, e.g., overall tumour burden.

In one aspect, there is provided a method for treating a subject presenting with CD47+ disease cells, comprising administering to the subject a pharmaceutical combination comprising an IgG4 isotype of SIRPαFc (designated SIRPαG4) and a CD38 antibody, such as daratumumab or its marketed form, Darzalex®. Suitably, the targeted disease cells are CD38+ and CD47+ in phenotype.

In a related aspect, there is provided the use of a SIRPαG4 in combination with a CD38 antibody for the treatment of a subject presenting with CD47+ disease cells such as cancer cells and especially cancer cells that have a CD47+/CD38+ phenotype.

In another aspect there is provided a pharmaceutical combination comprising a SIRPαG4 and a CD38 antibody for use in the treatment of CD47+/CD38+ disease cells.

There is also provided, in another aspect, a kit comprising a pharmaceutical combination comprising a SIRPαG4 and a CD38 antibody, together with instructions teaching their use in the treatment of disease cells.

In specific embodiments, the combination of the CD47 blocking agent and CD38 antibody is for use in the treatment of a blood cancer such as a myeloma, a lymphoma or a leukemia.

In alternative embodiments, the SIRPαFc used in combination with a CD38 antibody is a SIRPαG1. In other alternative embodiments, the CD38 antibody is daratumumab, or active, CD38-binding fragments thereof or active, CD38-binding variant, bispecific or bifunctional forms of daratumumab.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF REFERENCE TO THE DRAWINGS

FIG. 1 shows results when MM.1S (human multiple myeloma cell line) tumor bearing mice were treated with CD38 antibody daratumumab (10 mg/kg, 2×/week) in combination with SIRPαG4 (10 mg/kg, 5×/week) starting on day 11 post tumor inoculation, increased tumor growth inhibition (A and C) and improved survival (B) were observed compared to CD38 antibody daratumumab (10 mg/kg, 2×/week) monotherapy or SIRPαG4 (10 mg/kg, 5×/week) monotherapy; and

FIG. 2 shows results when Daudi (human Burkitt lymphoma cell line) tumor bearing mice were treated with CD38 antibody daratumumab (10 mg/kg, 2×/week) in combination with SIRPαG4 (10 mg/kg, 5×/week) starting on day 3 post tumor inoculation, increased tumor growth inhibition (A and C) and improved survival (B) were observed compared to CD38 antibody daratumumab (10 mg/kg, 2×/week) monotherapy or SIRPαG4 (10 mg/kg, 5×/week) monotherapy.

### DETAILED DESCRIPTION

The present disclosure provides methods, uses, combinations and kits useful for treating subjects that present with disease cells that have a CD47+ phenotype. In this method, CD47+ cancer subjects receive a combination of a CD38 antibody such as daratumumab, and a CD47 blocking agent which preferably is an Fc-fused form of human SIRPα, i.e., SIRPαFc, in which the Fc is an IgG4 isotype or an Fc receptor-binding variant thereof, designated SIRPαG4. The effect of the CD38 antibody is significantly enhanced by the CD47 binding SIRPαG4. The therapeutic effect is pronounced when the CD47+ disease cells are CD47+ cancer cells and tumours that will also bind daratumumab and thus have a CD38+ phenotype.

The term “CD47+” is used with reference to the phenotype of cells targeted for binding by the present CD47 blocking agents. Cells that are CD47+ can be identified by flow cytometry using CD47 antibody as the affinity ligand. CD47 antibodies that are labeled appropriately are available commercially for this use (for example, the antibody product of clone B6H12 is available from BD Biosciences). The cells examined for CD47 phenotype can include standard tumour biopsy samples including particularly blood samples taken from the subject suspected of harbouring endogenous CD47+ cancer cells. CD47 disease cells of particular interest as targets for therapy with the present drug combination are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry or by immunostaining or by gene expression analysis or the like, to be greater than the level measurable on a counterpart cell having a CD47 phenotype that is normal for that cell type.

The term “CD47+ disease cells” means cells that are associated with a disease and have a CD47+ phenotype. In one embodiment, the CD47+ disease cells are cancer cells.

In embodiments, the CD47 blocking agent is an IgG4 version of human SIRPαFc, which interferes with and dampens or blocks signal transmission that would result when CD47 interacts with SIRPα. As described in Trillium Therapeutics' WO2014/094122, the entire contents of which are incorporated herein by reference, the preferred SIRPαG4 is an Fc fused form of a region of human SIRPα that interacts with CD47 and has been shown to have anti-cancer activity. The term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. The present disclosure is directed most particularly to the drug combinations that include the human SIRP variant 2 form, or V2.

In the present drug combination, the SIRPαFc fusion protein has a SIRPα component that comprises at least residues 32-137 of human SIRPα (a 106-mer), which constitute and define the IgV domain of the V2 form according to current nomenclature. This SIRPα sequence, shown below, is referenced herein as SEQ ID No. 1.

[SEQ ID No. 1]
EELQVIQPDKSVSVAAGESAILHCTVTSLIPVGPIQWERGAGPARELIY
NQKEGHFPRVTTVSESTKRENMDESISISNITPADAGTYYCVKFRKGSP
DTEFKSGA

In a preferred embodiment, the SIRPαFc fusion protein incorporates the IgV domain as defined by SEQ ID No. 1, and additional, flanking residues contiguous within the SIRPα sequence. This preferred form of the IgV domain, represented by residues 31-148 of the V2 form of human SIRPα, is a 118-mer having the sequence shown below:

[SEQ ID No. 2]
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKPS.

The SIRPαFc protein incorporates an Fc region that has effector function. Fc refers to “fragment crystallisable” and represents the constant region of an antibody comprised principally of the heavy chain constant region and components within the hinge region. In embodiments, the Fc region includes the lower hinge-CH2-CH3 domains. More preferably, the Fc region includes the CH1-CH2-CH3 domains.

An Fc component “having effector function” is an Fc component having at least some natural or engineered function, such as at least some contribution to antibody-dependent cellular cytotoxicity or some ability to fix complement. Also, the Fc will at least bind to Fc receptors.

In embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank. In one specific and preferred embodiment, the G4 Fc region incorporates an alteration at position 228 (EU numbering), in which the serine at this position is substituted by a proline (S228P), thereby to stabilize the disulfide linkage within the Fc dimer.

In a specific embodiment, the Fc region is based on the amino acid sequence of a human IgG4 set out as P01861 in UniProtKB/Swiss-Prot, residues 99-327, and has the amino aid sequence shown below and referenced herein as SEQ ID No. 6:

[SEQ ID No. 3]
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVESCSVMHEALHNHYTQKSLSLSLGK

In an alternative embodiment, the SIRPαFc has an Fc region based on the amino acid sequence of a human IgG1 set out as P01857 in UniProtKB/Swiss, Prot residues 104-330, and has the amino acid sequence shown below:

[SEQ ID No. 4]
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK*

In a specific embodiment, when the Fc component is an IgG4 Fc, the Fc incorporates at least the S²²⁸P mutation, and has the amino acid sequence set out below and referenced herein as:

[SEQ ID No. 5]
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In a specific and preferred embodiment, the SIRPαFc fusion protein has the amino acid sequence number 6 set forth below: In this embodiment, the Fc component of the fusion protein is based on an IgG4, and incorporates the S²²⁸P mutation:

[SEQ ID No. 6]
EEELQVIQPDKSVSVAAGESAILHCTVTSLIPVGPIQWERGAGPARELI
YNQKEGHEPRVTTVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVESCSVMHEALHNHYTQKSLSL
SLGK

This SIRPαFc fusion protein is designated SIRPαG4.

In an alternative embodiment, the SIRPαFc fusion protein has the amino acid sequence set forth below: In this embodiment, the Fc component of the fusion protein is based on an IgG1:

[SEQ ID No. 7]
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

This SIRPαFc fusion protein is designated SIRPαG1.

In a preferred embodiment, the SIRPαFc protein is provided and used in a secreted homodimeric fusion form, in which two copies of the fusion protein are coupled through covalent binding between cysteines present in separate SIRPαFc single polypeptide chains, e.g. SIRPαG4 chains having SEQ ID No. 6.

The present drug combination comprises SIRPαG4, or SIRPαG1, as just described, and an antibody that binds cluster of differentiation 38, i.e., human CD38 (hCD38), also known as cyclic ADP ribose hydrolase. This is a glycoprotein found on the surface of many immune cells, including CD4+, CD8+, B lymphocytes and natural killer cells. CD38 also functions in cell adhesion, signal transduction and calcium signaling. It is a multifunctional ectoenzyme that catalyzes the synthesis and hydrolysis of cyclic ADP-ribose (cADPR) from NAD+ to ADP-ribose. These reaction products are essential for the regulation of intracellular Ca2+.

As used herein, the term “hCD38” refers to a protein that comprises the expressed and processed protein designated as UniProtKB/Swiss-Prot P28907. The term CD38 is used generically herein, and refers to the wild type protein and naturally occurring variants thereof. The term “wtCD38” is used more specifically with reference only to the wild type form of human CD38. The term “CD38+” is used to characterize the phenotype of disease cells that would bind to CD38 antibody and should respond to treatment with daratumumab. Targeted disease cells referred to herein as being “CD38+” include cancer cells that bind daratumumab, including cancer cells that over-express CD38, i.e., present with a greater density of surface CD38 than cells that are normal for CD38 or devoid of it.

Thus, a disease cell that has a CD47+/CD38+ phenotype is one that can bind with and respond to treatment with the CD47 blocking agent and the CD38 antibody.

The present combinations are based more particularly, and in one embodiment. on the hCD38 antibody known as daratumumab. now commercially available under the trade name Darzalex®. Daratumumab is a CD38-directed monoclonal antibody that binds to CD38, a signalling molecule highly expressed on the surface of multiple myeloma cells regardless of stage of disease. In doing so, daratumumab triggers the patient's own immune system to attack the cancer cells, resulting in rapid tumour cell death through multiple immune-mediated mechanisms of action and through immunomodulatory effects, in addition to direct tumour cell death via apoptosis (programmed cell death).

Daratumumab is an immunoglobulin G1 kappa (IgG1κ) human monoclonal antibody against CD38 antigen, produced in a mammalian cell line (Chinese Hamster Ovary). The molecular weight of daratumumab is approximately 148 kDa. In embodiments of the present invention, active fragments of daratumumab are used in the present combinations, instead of full length antibody. Useful fragments include particularly the Fab fragments.

In terms of amino acid sequence, daratumumab can be defined by its heavy and light chain sequences, reported at

http://www.genome.jp/dbget-bin/www_bget?dr:D10777, as follows:

Heavy chain
[SEQ ID No. 8]
EVQLLESGGG LVQPGGSLRL SCAVSGFTEN SFAMSWVRQA PGKGLEWVSA ISGSGGGTYY
ADSVKGRETI SRDNSKNTLY LQMNSLRAED TAVYFCAKDK ILWEGEPVED YWGQGTLVTV
SSASTKGPSV FPLAPSSKST SGGTAALGCL VKDYFPEPVT VSWNSGALTS GVHTFPAVLQ
SSGLYSLSSV VTVPSSSLGT QTYICNVNHK PSNTKVDKRV EPKSCDKTHT CPPCPAPELL
GGPSVFLFPP KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
EEMTKNQVSL TCLVKGFYPS DIAVEWESNG QPENNYKTTP PVLDSDGSFF LYSKLTVDKS
RWQQGNVESC SVMHEALHNH YTQKSLSLSP GK;
and
Light chain
[SEQ ID NO. 9]
EIVLTQSPAT LSLSPGERAT LSCRASQSVS SYLAWYQQKP GQAPRLLIYD ASNRATGIPA
RFSGSGSGTD FTLTISSLEP EDFAVYYCQQ RSNWPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC

Darzalex® is supplied as a colorless to pale yellow preservative-free solution for intravenous infusion in single-dose vials. The pH is 5.5. Darzalex is diluted with 0.9% Sodium Chloride Injection, USP. Each Darzalex® single-dose 20 mL vial contains 400 mg daratumumab, glacial acetic acid (3.7 mg), mannitol (510 mg), polysorbate 20 (8 mg), sodium acetate trihydrate (59.3 mg), sodium chloride (70.1 mg), and water for injection.

Each Darzalex® single-dose 5 mL vial contains 100 mg daratumumab, glacial acetic acid (0.9 mg), mannitol (127.5 mg), polysorbate 20 (2 mg), sodium acetate trihydrate (14.8 mg), sodium chloride (17.5 mg), and water for injection.

In one embodiment, the SIRPαG4 is used in combination with either a formulated daratumumab, or the already formulated Darzalex®.

Each drug included in the present pharmaceutical combination can be formulated separately for use in combination. The drugs are said to be used “in combination” when, in a recipient of both drugs, the effect of daratumumab enhances or at least influences the effect of the SRIPαG4. The drugs are in combination also when they are physically mixed for combined administration, and when they are placed separately within a kit that enables the present combination therapy.

The two drugs in the combination cooperate such that the effect of the combination is enhanced relative to either agent alone. In a preferred embodiment, the two drugs are used in combination to treat a cancer that has a phenotype that is CD47+/CD38+. This benefit manifests as a statistically significant improvement in a given parameter of target cell fitness or vitality. For instance, a benefit in CD47+ cancer cells, and especially in CD47+/CD38+ cancer cells, when exposed to a combination of CD47 blocking agent and CD38 antibody, could be a statistically significant decrease in the number of living cancer cells (hence a depletion), relative to non-treatment, or a decrease in the number or size of cancer cells or tumours, or an improvement in the endogenous location or distribution of any particular tumour type. The benefit could also be seen in terms of overall survival of treated subjects. In embodiments, the improvement resulting from treatment with the drug combination can manifest as an effect that is at least additive and desirably synergistic, relative to results obtained when only SIRPαG4 or only daratumumab is used. There can also be an improvement in the effectiveness of daratumumab on daratumumab resistant disease such as in advanced stage multiple myeloma patients or those with lower CD38 levels.

In use, each drug in the combination can be formulated as it would be for monotherapy, in terms of dosage size and form and regimen. In this regard, the improvement resulting from their combined use may permit the use of somewhat reduced dosage sizes or frequencies, as would be revealed in an appropriate clinical trial.

In this approach, each drug is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Each of the SIRPαG4 fusion protein and the CD38 antibody is formulated using practises standard in the art of therapeutics formulation. Solutions that are suitable for intravenous administration, such as by injection or infusion, are particularly useful.

Sterile solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” refers to an amount effective, at dosages and for a particular period of time necessary, to achieve the desired therapeutic result. A therapeutically effective amount of each drug in the combination may vary according to factors such as the disease state, age, sex, and weight of the individual, and the ability of the drug to elicit a desired response in the recipient. A therapeutically effective amount is also one in which any toxic or detrimental effects of the pharmacological agent are outweighed by the therapeutically beneficial effects. The CD38 antibody will of course be formulated in amounts that are suitable for patient dosing, as permitted by the regulatory agencies that have approved its use in humans. In use, each drug in the combination thus is formulated as it would be for monotherapy, in terms of dosage size and form and regimen. In this regard, the cooperation/benefit resulting from their combined use may permit the use of somewhat reduced dosage sizes or frequencies, as would be revealed in an appropriately controlled clinical trial.

The SIRPαFc fusion protein can be administered to the subject through any of the routes established for protein delivery, in particular intravenous, intradermal, intratumoural and subcutaneous injection or infusion, or by oral or nasal administration.

The drugs in the present combination can be administered sequentially or, essentially at the same time, e.g., consecutively or concurrently. In embodiments, the CD38 antibody is given before administration of the SIRPαFc. In the alternative, the CD38 antibody can be given after or during administration of the SIRPαFc. Thus, in embodiments, the subject undergoing therapy is a subject already treated with one of the combination drugs, such as the CD38 antibody, that is then treated with the other of the combination drugs, such as the SIRPαFc drug. Most desirably, the activities of the two drugs overlap within the patient for a period sufficient to gain the improvement in activity fostered when the drugs are used in combination.

Dosing regimens are adjusted to provide the optimum desired response (e.g., a therapeutic response). For example, a single bolus of each drug may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier.

The drugs can be formulated in combination, so that the combination can be introduced to the recipient in one administration, e.g., one injection or one infusion bag. Alternatively, the drugs can be combined as separate units that are provided together in a single package, and with instructions for the use thereof according to the present method. In another embodiment, an article of manufacture containing the SIRPαFc drug and CD38 antibody combination in an amount useful for the treatment of the disorders described herein is provided. The article of manufacture comprises one or both drugs of the present antibody drug combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used in combination with SIRPαFc drug in accordance with the present disclosure, thereby to elicit an enhanced effect on the CD47+ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

For administration the dose for the SIRPαFc drug will be within the range from about 0.0001 to 100 mg/kg, and more usually 0.01 to 10 mg/kg, of the host body weight. For example, parenteral SIRPαFc dosages can be 0.3 mg/kg body weight, 1 mg/kg body weight, 3 mg/kg body weight, 5 mg/kg body weight or 10 mg/kg body weight or within the range of 0.1-100 mg/kg. When the CD47 blocking agent is a SIRPαFc fusion protein of SEQ ID No. 6 or 7, the dose can be about 1 ug-10 mg per dose administered by intratumoural injection.

Daratumumab dosing is established for the treatment of multiple myeloma, and this same approach can guide its use for the treatment of other indications as well. That is, daratumumab is indicated as monotherapy for multiple myeloma in patients who have received at least 3 lines of therapy, including a proteasome inhibitor (PI) and an immunomodulatory agent (IMiD), or who are double-refractory to a PI and IMiD. Weeks 1-8: 16 mg/kg IV infusion once weekly; Weeks 9-24: 16 mg/kg IV infusion every 2 weeks; and Week 25 onward until disease progression: 16 mg/kg IV infusion every 4 weeks.

Daratumumab is also indicated in combination with bortezomib and dexamethasone for the treatment of patients with multiple myeloma who have received at least 1 prior therapy: weeks 1-9: 16 mg/kg IV infusion once weekly; weeks 10-24: 16 mg/kg IV infusion every 3 weeks; and week 25 onwards until disease progression: 16 mg/kg IV infusion every 4 weeks.

Daratumumab is also indicated in combination with lenalidomide and dexamethasone for the treatment of patients with multiple myeloma who have received at 1 prior therapy Weeks 1-8: 16 mg/kg IV infusion once weekly; Weeks 9-24: 16 mg/kg IV infusion every 2 weeks; and Week 25 onwards until disease progression: 16 mg/kg IV infusion every 4 weeks

As well, daratumumab treatment is indicated in combination with pomalidomide and dexamethasone for the treatment of patients with multiple myeloma who have received at least 2 prior therapies including lenalidomide and a proteasome inhibitor; weeks 1-8: 16 mg/kg IV infusion once weekly; weeks 9-24: 16 mg/kg IV infusion every 2 weeks; and Week 25 onwards until disease progression: 16 mg/kg IV infusion every 4 weeks.

Daratumumab (JNJ-54767414) can be administered as an intravenous (IV) infusion at a dose of 16 mg/kg weekly for the first 3 cycles, on Day 1 of Cycles 4-8 (every 3 weeks), and then on Day 1 of subsequent cycles (every 4 weeks). First 8 Cycles are 21-day cycles; Cycles 9 and onwards are 28-day cycles.

As noted daratumumab can be used in combination with a proteasome inhibitor known as bortezomib and with dexamethasone.

Bortezomib can be administered at 1.3 mg/m2 subcutaneously (sc) on Days 1, 4, 8 and 11 of each 21-day cycle. Eight Bortezomib treatment cycles can be administered.

Dexamethasone can be administered orally at 20 mg on Day 1, 2, 4, 5, 8, 9, 11 and 12 of the first 8 bortezomib treatment cycles (except for Cycles 1-3). In Cycles 1-3, participants receive dexamethasone 20 mg on days 1, 2, 4, 5, 8, 9, 11, 12 and 15. During weeks when the participant receives an infusion of daratumumab, dexamethasone will be administered at a dose of 20 mg IV before the daratumumab infusion as pre-infusion medication.

The drug combination is useful to treat a variety of CD47+ disease cells. In one embodiment, the drug combination can used to inhibit the growth or proliferation of cells that are CD47+ and DC38+. These cancers include solid cancers including carcinoma and sarcomas, as well as hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS): and myelodysplastic syndrome. “Lymphoma” may refer to a Hodgkin's lymphoma, both indolent and aggressive non-Hodgkin's lymphoma, cutaneous T cell lymphoma (CTCL), peripheral T cell lymphoma (PTCL) Burkitt's lymphoma, Mantle cell lymphoma (MCL) and follicular lymphoma (small cell and large cell), among others. Myelomas include multiple myeloma (MM), giant cell myeloma, heavy-chain myeloma, and light chain myeloma and Bence-Jones myeloma.

In some embodiments, the hematological cancer treated with the drug combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the drug combination is a CD47+ lymphoma or myeloma selected from Hodgkin's lymphoma, both indolent and aggressive non-Hodgkin's lymphoma, diffuse large cell lymphoma (DLBCL), mantle cell lymphoma. T cell lymphoma including mycosis fungoides, Sezary's syndrome, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

In a specific embodiment, the cancer treated with the present combination is multiple myeloma. In another specific embodiment, the targeted cancer is mantle cell lymphoma. In a further embodiment, the cancer treated with the present combination is relapsed or refractory Hodgkin's lymphoma. In another specific embodiment, the CD47 blocking agent is SIRPαG4. In a further specific embodiment the CD38 antibody is daratumumab.

In still other embodiments, daratumumab is used in combination with SIRPαFc, such as SEQ ID No. 6 or SEQ ID No. 7, such as for the treatment of cutaneous T cell lymphoma or multiple myeloma. In another embodiment, the combination is used to treat a T cell lymphoma such as mycosis fungoides or Sezary's syndrome.

Thus, in specific embodiments, there is provided the use of a CD47 blocking agent in combination with an CD38 antibody for the treatment of a particular CD47+ cancer, wherein.
i) the CD47 blocking agent is SIRPαG4 of SEQ ID No. 1 and the CD38 antibody is daratumumab, such as for the treatment of a cancer that is cutaneous T cell lymphoma or multiple myeloma or relapsed or refractory Hodgkin's lymphoma;
ii) the CD47 blocking agent is SIRPαG1 of SEQ ID No. 2 and the CD38 antibody is daratumumab, such as for the treatment of a cancer that is cutaneous T cell lymphoma or multiple myeloma or relapsed or refractory Hodgkin's lymphoma;
iii) the CD47 blocking agent is any SIRPαFc and the CD38 antibody is daratumumab, such as for the treatment of a cancer that is cutaneous T cell lymphoma or multiple myeloma.

Other cancers that can be treated with a combination of SIRαG4 and daratumumab include those having a CD38+/CD47+ phenotype. Cancers that can be targeted for treatment include solid tumours including Merkel cell carcinoma, hematologic malignancies such as monoclonal gammopathy, smoldering multiple myeloma, mantel cell lymphoma, diffuse large B-cell lymphoma, follicular lymphoma, non-Hodgkin's lymphoma, acute myeloid leukemia, acute lymphoblastic leukemia and chronic lymphocytic leukemia.

Desirable pharmaceutical combinations will show a statistically significant improvement in cancer cell response. This can be demonstrated as a statistically significant improvement in CD38 antibody activity caused by combination with a CD47 blocking agent, or vice versa, where statistical significance is shown as noted in the examples that follow and desirably. provides a p value >0.05 and more desirably >0.01 such as >0.001.

The combination therapy, comprising CD47 blockade and CD38 inhibition can also be exploited together with any other agent or modality useful in the treatment of the targeted indication, such as surgery as in adjuvant therapy, or with additional chemotherapy as in neoadjuvant therapy. Daratumumab in particular can be used with lenalidomide, bortezomib and dexamethasone, in the manner approved for the treatment of patients with multiple myeloma.

The following non-limiting examples illustrate the present disclosure.

### Examples

Tumor cells frequently evade macrophage-mediated destruction by increasing cell surface expression of CD47, which delivers an anti-phagocytic (“do-not-eat”) signal by binding the inhibitory signal regulatory protein a (SIRPα) receptor on macrophages. Previous studies have shown that blockade of the CD47-SIRPα pathway using TTI-621, a soluble SIRPα-IgG1 Fc fusion protein, triggers macrophage phagocytosis of tumor cells in vitro, and potently inhibits tumor growth in vivo. In the current study, the in vitro and in vivo efficacy of SIRPαG4 (SEQ ID No. 6), a soluble SIRPα-Fc variant protein containing an IgG4 Fc tail, was evaluated in multiple model systems.

Unlike CD47-blocking antibodies, SIRPαG4 binds minimally to human erythrocytes, and does not induce hemagglutination in vitro. Therefore, it avoids a large circulating antigen sink, and is less likely to cause anemia in patients. Additionally, SIRPαG4 potently induces phagocytosis of a broad panel of tumor cells derived from patients with both hematological and solid tumors. Although in vitro phagocytosis of human platelets is also observed, SIRPαG4 preferentially induces phagocytosis of tumor cells over platelets in a competitive phagocytosis assay.

The in vivo efficacy of SIRPαG4 monotherapy and/or combination therapy was evaluated in different tumor models. In the Burkitt lymphoma (Daudi) and multiple myeloma (MM.1S) xenograft tumor models, the potential of combining SIRPαG4 with daratumumab (anti-CD38 antibody) was also explored. In both models, SIRPαG4 monotherapy demonstrated partial tumor growth inhibition. However, the therapeutic efficacy was further enhanced when SIRPαG4 was combined with daratumumab.

Collectively, these results demonstrate that SIRPαG4 induces potent, tumor-specific macrophage phagocytosis across a range of hematological and solid tumors, and is efficacious as a monotherapy agent in a DLBCL xenograft tumor model. Furthermore, SIRPαG4 potentiates the efficacy of daratumumab in hematological xenograft tumor models. These data support the use of SIRPαG4 in combination with anti-tumor antibodies in cancer patients with hematological malignancies.

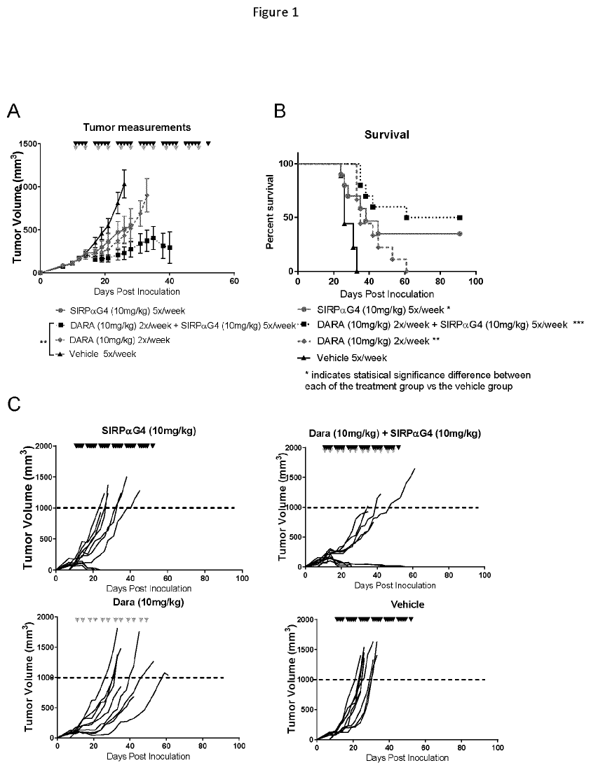

In particular, as shown in FIG. 1, 5×10⁶ MM.1S cells (dexamethasone sensitive multiple myeloma) in Matrigel were implanted subcutaneously into the right flank of NOD SCID (n=9-10 mice per group) on day 0. Mice were randomized on day 11 when the mean tumor size was approximately 112-114 mm³ and received intraperitoneal (IP) injections of SIRPαG4 10 mg/kg 5×/week (Black triangles) or/and Daratumumab 10 mg/kg 2×/week (Gray triangles) or vehicle control 5×/week (not shown). FIG. 1A shows the mean tumor volume with standard mean deviation of each treatment group is shown. The curve terminates when ≥25% of animals per group were sacrificed. Statistical significance was calculated by one-way ANOVA (Tukey's multiple comparisons test) based on tumor volumes on day 26. FIG. 1B reveals the benefit in terms of enhanced survival of the tumor bearing mice. Statistical significance of the survival curves was calculated by LogRank test (adjusted for multiple comparison) using Prism GraphPad software. FIG. 1C provides individual tumor growth spider plot of each treatment group. The dosing schedules are indicated as inverted triangles.

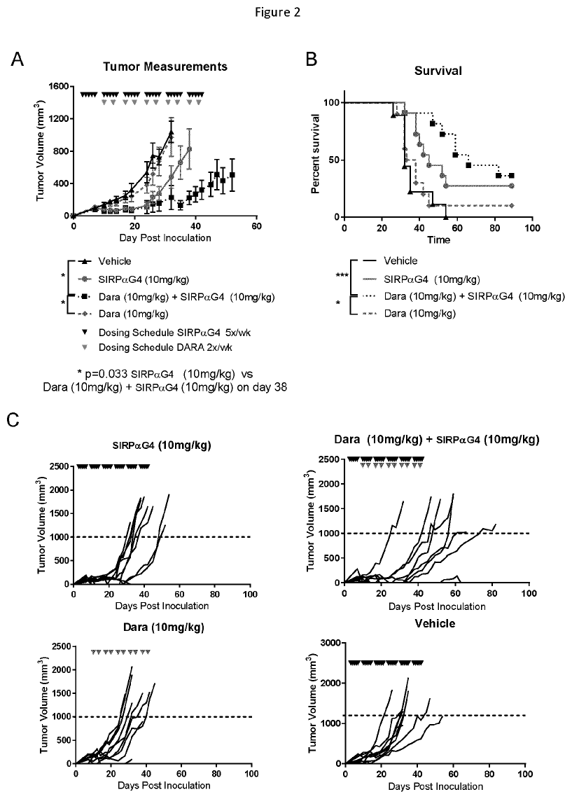

Also, and as shown in FIG. 2, 1×10⁷ Daudi cells (a B lymphoblastic cell line from a 16 year old male; positive for EBNA, carries EBV markers, complement receptors, surface bound immunoglobulin and surface markers for the Fc fragment of IgG) in Matrigel were implanted subcutaneously into the right flank of NOD SCID. Mice were treated with intraperitoneal (IP) injections of 10 mg/kg SIRPαG4 5×/week starting on day 3 (Black triangles) or/and 10 mg/kg Daratumumab 2×/week starting on day 10 (Gray triangles) or vehicle control 5×/week (not shown). (FIG. 2A) The mean tumor volume with standard mean deviation of each treatment group is shown. The curve terminates when ≥25% per group were sacrificed. Statistical significance was calculated by one-way ANOVA (Tukey's multiple comparisons test) based on tumor volumes on day 32. FIG. 2B reveals enhancement of survival of the tumor bearing mice. Statistical significance of the survival curves was calculated by LogRank test (adjusted for multiple comparisons) using Prism GraphPad software. FIG. 2C provides individual tumor growth spider plots of each treatment group. The dosing schedules are indicated as inverted triangles.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A use of a combination of a SIRPαFc protein and a CD38 antibody for treating a subject presenting with disease cells.

2. The use according to claim 1, wherein the CD38 antibody is daratumumab.

3. (canceled)

4. The use according to claim 1, wherein the SIRPαFc drug comprises SEQ ID No. 6.

5. The use according to claim 1, wherein the SIRPαFc drug comprises SEQ ID No. 7.

6. The use according to claim 1, wherein the disease cells are cancer cells having a CD47+/CD38+ phenotype.

7. The use according to claim 6, wherein the cancer cells are blood cancer cells or solid tumour cells.

8. Thus use according to claim 7, wherein the cancer cells are blood cancer cells.

9. The use according to claim 8, wherein the blood cancer cell is a leukemia, a lymphoma or a myeloma.

10. The use according to claim 9, wherein the disease cells are cells of a cancer type selected from acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome.

11. The use according to claim 10, wherein the cancer is a lymphoma selected from a Hodgkin's lymphoma, both indolent and aggressive non-Hodgkin's lymphoma, Burkitt's lymphoma, and follicular lymphoma (small cell and large cell).

12. The use according to claim 10, wherein the cancer is a myeloma selected from multiple myeloma, giant cell myeloma, heavy-chain myeloma, and light chain or Bence-Jones myeloma.

13. The use according to claim 1, wherein the CD38 antibody is for use in a subject that has already received the SIRPαFc drug.

14. A pharmaceutical combination comprising an effective amount of a SIRPαFc drug, and an effective amount of a CD38 antibody.

15. The combination according to claim 14, wherein the CD38 antibody is daratumumab.

16. (canceled)

17. The combination of claim 14, wherein the SIRPαFc drug comprises SEQ ID No. 6.

18. The combination of claim 14, wherein the SIRPαFc drug comprises SEQ ID No. 7.

19. A kit comprising a combination of claim 1, and written instructions for the use thereof for the treatment of a subject presenting with CD47+ disease cells.


SEQUENCE LISTING (9 sequences)

SEQ ID NO: 1 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 3 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 345 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC